(19) United States
(12) Patent Application Publication
(10) Pub. No.: US 2015/0208952 A1
(43) Pub. Date: Jul. 30, 2015

(54) BREATH SAMPLING TUBES

(71) Applicant: ORIDION MEDICAL 1987 LTD., Jerusalem (IL)

(72) Inventor: Paul S. Addison, Midlothian (GB)

(73) Assignee: ORIDION MEDICAL 1987 LTD., Jerusalem (IL)

(21) Appl. No.: 14/167,774

(22) Filed: Jan. 29, 2014

(51) Int. Cl. A61B 5/097; A61M 16/08

(52) U.S. Cl. CPC A61B 5/097; A61M 16/0808

(57) ABSTRACT

The present disclosure provides breath sampling tubes having an outer wall and an inner wall, wherein the outer wall includes at least one groove having uneven side walls arranged to induce a convection driven circulation zone in the groove; wherein at least part of the tube is formed of a material configured to evaporate liquid.

### TECHNICAL FIELD

The present disclosure generally relates to the field of breath sampling tubes.

### BACKGROUND

Accurate monitoring concentrations of a gas, such as for example carbon dioxide (CO₂) in exhaled breath, is vital in assessing the physiologic status of a patient. Breath sampling is generally performed through breath sampling tubes configured to be connected to a patient airway and to a medical device.

Liquids are common in patient sampling systems, and have several origins, for example condensed out liquids from the highly humidified air provided to and exhaled from the patient. These liquids typically accumulate both in the patient airway and in the sampling line tubing; secretions from the patient, typically found in the patient airway; and medications or saline solution provided to the patient during lavage, suction and nebulization procedures.

### SUMMARY

The present disclosure relates to breath sampling tubes including an outer wall having at least one groove with uneven side walls. The breath sampling tubes disclosed herein are configured to evaporate liquids.

One of the major obstacles when designing a filter system is the necessity to prevent any liquids from blocking the breath sampling path or from reaching the measurement sensor while providing continuous, smooth, undisturbed sampling of the patient's breath.

A well-known problem with gas sampling lines is that they may eventually saturate allowing the line to become clogged. Lines are designed so that water vapor is captured and evaporated through the tube surface. At high humidity, however, the evaporation flow rate may be less than the capture rate and so eventually the reservoir, or other suitable liquid collection element, saturates and the line may become clogged. The time it takes for this to happen is known as the lifetime of the line.

The breath sampling tubes disclosed herein, includes an outer wall and an inner wall, wherein at least a part of the outer wall has at least one groove. The groove has uneven side walls arranged to induce a convection driven circulation zone in the groove due to increased air flow instability. The circulation zones cause air to be driven over the surface of the tube thereby increasing the evaporation rate of water vapor at the surface-air interface. As a result, the lifetime of the line is extended.

The formation of the groove(s) in the surface of the tube may further serve to influence the rigidity of the tube and the ease of manufacturing.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

According to some embodiments, there is provided a breath sampling tube having an outer wall and an inner wall, wherein the outer wall includes at least one groove having uneven side walls arranged to induce a convection driven circulation zone in the at least one groove; wherein at least part of the tube is formed of a material configured to evaporate liquid.

According to some embodiments, the uneven side walls include a first side wall and a second side wall, wherein the length (dl) of the first side wall is different from the length (d2) of the second side wall. According to some embodiments, (d1)>(d2). According to some embodiments, (d1)<(d2).

According to some embodiments, the at least one groove is formed circumferentially around the tube.

According to some embodiments, the outer wall includes a plurality of grooves having uneven side walls.

According to some embodiments, the at least one groove generates a rough surface in the outer wall.

According to some embodiments, the at least one groove enhance airflow instability along the outer wall of the tube. According to some embodiments, the at least one groove enhances the evaporation of liquids from the tube, thereby extending the life time of the tube.

According to some embodiments, the material configured to evaporate liquids is a hydrophilic material. According to some embodiments, the outer wall includes a hydrophilic material. According to some embodiments, the inner wall includes a hydrophilic material.

According to some embodiments, the breath sampling tube further includes an inner conduit. According to some embodiments, the inner conduit is configured to permit gas flow along a central portion of the conduit and to store liquids along a surface of the conduit. According to some embodiments, the inner conduit includes a hydrophilic material.

According to some embodiments, there is provided a breath sampling system including: a breath sampling tube having an outer wall and an inner wall wherein at least part of the outer wall includes at least one groove having uneven side walls arranged to induce a convection driven circulation zone in the at least one groove; wherein at least part of the tube is formed of a material configured to evaporate liquids; and at least one connector.

According to some embodiments, there is provided a method including forming a breath sampling tube having an outer wall and an inner wall, wherein at least a part of the outer wall includes at least one groove having uneven side walls arranged to induce a convection driven circulation zone in the at least one groove.

According to some embodiments, at least part of the tube is formed of a material configured to evaporate water.

According to some embodiments, the uneven side walls include a first side wall and a second side wall, wherein a length (dl) of the first side wall is different from a length (d2) of the second side wall.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1A schematically illustrates evaporation through a surface of a tube, according to some embodiments;

FIG. 1B schematically illustrates the lifetime of tube lines against the vapor pressure of water (P_{H2O}) in prior art tube lines (A), and for the tube lines disclosed herein (B), according to some embodiments;

FIG. 2 schematically illustrates convection driven circulation zones generated in grooves having uneven side walls, according to some embodiments;

FIG. 3A schematically illustrates a tube with grooves having uneven side walls, according to some embodiments;

FIG. 3B schematically illustrates a tube with grooves having uneven side walls, according to some embodiments;

FIG. 3C schematically illustrates a tube with grooves having uneven side walls, according to some embodiments;

FIG. 3D schematically illustrates a tube with grooves having uneven side walls, according to some embodiments;

FIG. 3E schematically illustrates a tube with grooves having uneven side walls, according to some embodiments;

FIG. 4 schematically illustrates a tube with non-successive grooves with uneven side walls, according to some embodiments;

FIG. 5A schematically illustrates a tube with grooves having uneven side walls in the entire length thereof, according to some embodiments;

FIG. 5B schematically illustrates a tube with grooves having uneven side walls at a distal end thereof, according to some embodiments;

FIG. 5C schematically illustrates a tube with grooves having uneven side walls at a proximal end thereof, according to some embodiments;

FIG. 5D schematically illustrates a tube with grooves having uneven side walls at a central portion thereof, according to some embodiments;

FIG. 5E schematically illustrates a tube with grooved sectioned along the tubing line, according to some embodiments;

FIG. 6 schematically illustrates a tube with grooves having uneven side walls and with an inner conduit, according to some embodiments;

FIG. 7 schematically illustrates a breath sampling system, according to some embodiments.

FIG. 8 schematically illustrates a breath sampling system, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

There is provided, according to some embodiments, a tube including an outer wall and an inner wall, wherein at least a part of the outer wall includes at least one groove having uneven side walls arranged to induce a convection driven circulation zone in the at least one groove. According to some embodiments, at least part of the tube is formed of a material configured to evaporate fluids.

According to some embodiments, the tube is a breath sampling tube. According to some embodiments, the tube is part of a breath sampling tube. According to some embodiments, the tube is configured to be connected to a breath sampling tube. The tubes disclosed herein may be integrally formed with a commonly used breath sampling tube or be a separate element (and/or an “add-on”) which may be attached to a breath sampling tube, for examples by adapter(s) and or connector(s).

As used herein, the terms “breath sampling tube”, “sampling line” and “breath sampling line” may refer to any type of tubing(s) or any part of tubing system adapted to allow the flow of sampled breath, for example, to an analyzer, such as a capnograph. The sampling line may include tubes of various diameters, adapters, connectors, valves, drying elements (such as filters, traps, drying tubes, such as Nafion® and the like).

As used herein, the term “at least a part of” may refer to the entire tube, the proximal end of the tube, the distal end of the tube, a central part of the tube, in proximity to a liquid trap or reservoir, at a certain distance from a liquid trap or reservoir, as sections along the tube or any other suitable part of the tube line. Each possibility is a separate embodiment.

As used herein, the terms “distal” and “distal end” may refer to the part of the tube closest to the subject. The length of the distal end may for example be 0.5, 1, 2, 3, 4, 5, 10 cm or more, of the tube. Each possibility is a separate embodiment.

As used herein, the terms “proximal” and “proximal end” may refer to the part of the tube closest to the medical device. The length of the proximal end may for example be 0.5, 1, 2, 3, 4, 5, 10 cm or more, of the tube. Each possibility is a separate embodiment.

As used herein, the term “close proximity” may refer to 30, 20, 15, 10, 5, 1, 0.5 cm or less. Each possibility is a separate embodiment.

As used herein, the term “certain distance” may refer to a distance larger than 10 cm, for example larger than 20 cm, 30 cm, 40 cm or 50 cm, 70 cm. Each possibility is a separate embodiment.

As used herein, the term “groove(s)” may refer to a channel or a furrow formed in the outer wall of the breath sampling tube.

As used herein, the term “at least one groove” may refer to one groove, 2 grooves, 3 grooves, 4 grooves, 5 grooves, 10 grooves, 100 grooves or more, any number there between or any other suitable number of grooves. Each possibility is a separate embodiment. For example, according to some embodiments, the outer wall comprises at least 3 grooves. For example, according to some embodiments, the outer wall comprises at least 10 grooves.

According to some embodiments, the outer wall comprises a plurality of grooves. According to some embodiments, the plurality of grooves generates a rough surface in the outer wall. According to some embodiments, the plurality of grooves is identical. According to some embodiments, at least part of the plurality of grooves is not identical.

According to some embodiments, the term “uneven side walls”, as used herein, may refer to grooves having side walls of different lengths. According to some embodiments, the length of the side wall may be the length between the point of the side wall closest to the inner wall of the tube and the point of the side wall furthest away from the inner wall of the tube. According to some embodiments, the term “uneven side walls”, as used herein, may refer to grooves having side walls of different angles relative to the axis of the tube. According to some embodiments, the term “uneven side walls”, may refer to grooves having side walls of different heights. According to some embodiments, the height of the side wall may be the distance from the highest point of the side wall to the inner wall of the tube. According to some embodiments, the term “uneven side walls”, may refer to grooves having side walls of different shape. According to some embodiments, the uneven side walls of the groove(s) may produce a convection driven recirculation zone in the groove. The recirculation zones may cause air to be driven over the surface of the tube thereby increasing the evaporation rate of water vapor at the surface-air interface. Therefore, under normal vapor pressure of water (P_{H2O}) conditions, the tube of the present disclosure may evaporate more liquids, thereby avoid blockage of the tube and in effect extend its lifetime.

According to some embodiments, the groove(s) may be replaced by a ridge(s)/elevation(s), which likewise may produce a recirculation zone, and as such fall under the scope of this disclosure.

According to some embodiments, the material configured to evaporate liquids is a hydrophilic material. According to some embodiments, the outer wall includes a hydrophilic material. According to some embodiments, the inner wall includes a hydrophilic material. According to some embodiments, the hydrophilic material is a hydrophilic wicking material such as a porous plastic having a pore size ranging from approximately 5 microns to approximately 50 microns.

According to some embodiments, the at least one groove, having uneven side walls, may include a first side wall and a second side wall, wherein a length (d1) of the first side wall is different from a length (d2) of the second side wall. According to some embodiments (d1) is longer than (d2). According to some embodiments, (d1) is shorter than (d2). For example, (d1) may be twice the length of (d2). For example, the (d1) may be ⅓ the length of (d2). For example, the ratio of (d1) to (d2) may be in the range of 0.25-0.85, in the range of 1:1.25-1:5 or any other suitable ratio. Each possibility is a separate embodiment.

According to some embodiments, the at least one groove having uneven side walls may include a wall vertical to the inner wall of the tube and a wall sloped relative to the inner wall of the tube, such that the angle between the vertical wall and the sloped wall is less than 90°, for example but not limited to 10°-85°, 25°-65° or 30°-60°. Each possibility is a separate embodiment. Alternatively, according to some embodiments, the at least one groove having uneven side walls may have two sloped walls of different angles relative to an inner wall of the tube, such that the angle between the slopes, generating the groove, is larger than 90°, for example but not limited to an angle in the range of 100°-175°, 120-165° or 130°-160°. Each possibility is a separate embodiment. Alternatively, according to some embodiments, the at least one groove having uneven side walls may have two sloped walls of different angles relative to an inner wall of the tube, such that the angle between the slopes, generating the groove, is less than 90°, for example but not limited to an angle in the range of 10°-85°, 25°-65° or 30°-60°. Each possibility is a separate embodiment.

According to some embodiments, the at least one groove having uneven side walls may have sloped walls of different heights. According to some embodiments, the at least one groove, having uneven side walls, may include a first side wall and a second side wall, wherein the height (h1) of the first side wall is bigger than the height (h2) of the second side wall. According to some embodiments, (h1) is higher than (h2). According to some embodiments, (h1) is lower than (h2). For example, (h1) may be twice the height of (h2) of the second side wall. For example, (h1) may be ⅓ the height of (h2). For example, the ratio of (h1) to (h2) may be in the range of 0.25-0.85, in the range of 1:1.25-1:5 or any other suitable ratio. Each possibility is a separate embodiment.

According to some embodiments, the grooves having uneven side walls may be consecutive along or around the tube, such that two successive grooves share a wall. Alternatively, the grooves may not be immediately successive, but rather be separated, such that each groove has it separate walls. For example, each groove may be separated at least 1 mm, at least 5 mm, at least 1 cm, at least 5 cm or more from its closest neighboring groove. Each possibility is a separate embodiment.

According to some embodiments, the at least one groove may have a depth in the range of 0.005 mm-1 mm. According to some embodiments, the at least one groove may have a depth in the range of 0.01 mm to 0.5 mm in the outer wall of the tube.

According to some embodiments, the at least one groove having uneven side walls may be formed parallel to a main axis of a tube (longitudinally), such as for example a breath sampling tube. Alternatively, according to some embodiments, the at least one groove having uneven side walls may be formed orthogonal to a main axis of the tube (circumferentially). Alternatively, according to some embodiments, the at least one groove having uneven side walls may be formed helically to a main axis of the tube. Alternatively, according to some embodiments, the at least one groove having uneven side walls may be formed unevenly on the outer wall of the tube. It is understood by one of ordinary skill in the art, that the pattern of the groove(s) on the tube may influence the flexibility of the tube. For example, circumferential groove(s) or helical groove(s) around the tube may form a tube with greater flexibility as compared to a tube with longitudinal groove(s) or as compared to a tube without groove(s). Such flexibility may be important both in use and in efficient packaging and storage.

According to some embodiments, the tube further comprises an inner conduit. According to some embodiments, at least a portion of the inner conduit is non-cylindrical and configured to store liquids. According to some embodiments, the inner conduit is configured to permit gas flow along a central portion of the conduit and to store liquids along a surface of the conduit. According to some embodiments, the surface of the inner conduit comprises a hydrophilic material. According to some embodiments, the inner conduit may include a first lumen and a second lumen. According to some embodiments, the diameter of the first lumen is larger than the diameter of the second lumen. According to some embodiments, the inner conduit may be adapted to collect liquids in the first lumen and to permit gas flow in the second lumen. According to some embodiments, the surrounding surface of the first lumen may be more hydrophilic than the surrounding surface of the second lumen.

There is provided, according to some embodiments, a breath sampling system including: a breath sampling tube having an outer wall and an inner wall wherein at least part of the outer wall comprises at least one groove having uneven side walls arranged to induce a convection driven circulation zone in the at least one groove and at least one connector. According to some embodiments at least part of the tube is made from a material configured to evaporate liquids. It is understood that the tube of the breath sampling system may be the tube described in any one or more of the above embodiments.

According to some embodiments, the connector is molded on the breath sampling tube. According to some embodiments, the connector is a separate element configured to be attached to the breath sampling tube.

According to some embodiments, the connector is configured to connect between the breath sampling tube and a patient airway tubing. According to some embodiments, the connector is configured to connect to an oral/nasal cannula.

According to some embodiments, the system further comprises a moisture reduction system, hereinafter referred to as MRS. The MRS may be a specially designed tube, which may be of variable length and diameter, adapted to reduce moisture entering the breath sampling tube. The MRS may include any drying mechanism and/or material, essentially impermeable to gas, that is capable of reducing moisture level, such as but not limited to a Nafion® tube. According to some embodiments, the system may further include filters such as micro-porous filters or molecular sieves (material containing tiny pores of a precise and uniform size that may be used to absorb moisture). According to some embodiments, the system may further include a liquid trap and/or reservoir configured to trap liquids in the sampling tube. According to some embodiments the system may further comprise a medical device such as but not limited to a capnograph.

There is provided, according to some embodiments, a breath sampling system including: a breath sampling tube and an oral nasal cannula. According to some embodiments the tube includes an outer wall and an inner wall wherein at least part of the outer wall has at least one groove. According to some embodiments, the at least one groove has uneven side walls arranged to induce a convection driven circulation zone in the at least one groove. According to some embodiments, at least part of the tube is made from a material configured to evaporate liquids. It is understood that the tube of the breath sampling system may be the tube described in any one or more of the above embodiments.

According to some embodiments, the oral/nasal cannula is an integral part of the breath sampling tube. According to some embodiments, the oral/nasal cannula is molded on the breath sampling tube. According to some embodiments, the oral/nasal cannula is a separate element configured to be attached to the breath sampling tube.

According to some embodiments, the system further comprises a moisture reduction system, as essentially described above.

According to some embodiments, there is provided a method including forming a breath sampling tube having an outer wall and an inner wall, wherein at least a part of the outer wall includes at least one groove having uneven side wall arranged to induce a convection driven circulation zone in the at least one groove. According to some embodiments, at least part of the tube is formed of a material configured to evaporate liquids. It is understood that the at least one groove may have any distribution and/or configuration in, along and/or around the sampling tube, as essentially described above. For example the at least one groove having uneven side walls may include a first side wall and a second side wall, wherein the length (d1) of the first side wall is different from the length (d2) of the second side wall. For example the at least one groove may have two sloped walls of different angles relative to an inner wall of the tube, such that the angle between the slopes is different from 90°. For example, the at least one groove having uneven side walls may have a first side wall and a second side wall, wherein a height (h1) of the first side wall is different from height (h2) of the second side wall. For example the at least one groove may be formed circumferentially, longitudinally or helically in the outer wall of the tube. For example the at least one groove may be formed along the entire length of the tube or in parts thereof. According to some embodiments each of the grooves may be identical. Alternatively, at least some of the grooves may be non-identical.

Furthermore the number of grooves made may be any suitable number, as essentially described above. According to some embodiments, the at least one groove having uneven side walls may be replaced by at least one ridge or elevation with uneven sidewalls, which likewise serves to produce recirculation zones as essentially described above, and as such fall under the scope of this disclosure.

There is provided, according to some embodiments, a method for breath sampling including: channeling breath through a breath sampling tube, the breath sampling tube having an outer wall and an inner wall wherein at least part of the outer wall comprises at least one groove having uneven side wall arranged to induce a convection driven circulation zone in the at least one groove; wherein at least part of the tube is made from a material configured to evaporate liquids. It is understood that the tube of the method may be the tube described in any one or more of the above embodiments.

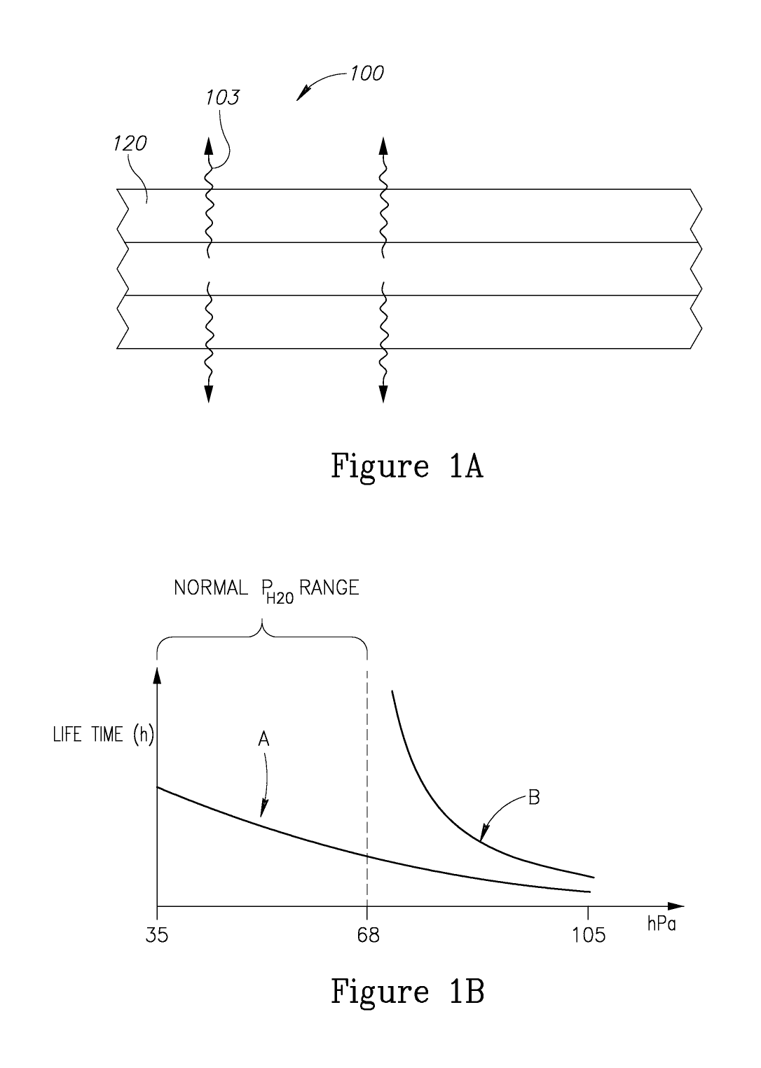

Reference is now made to FIG. 1A, which schematically illustrates evaporation through a surface of a tube 100, according to some embodiments. Tube 100, may for example be a breath sampling tube, and is generally configured to allow water to evaporate (shown as arrows 103) through an outer wall 120 of tube 100. According to some embodiments, outer wall 120 is made of a hydrophilic material, such as for example a hydrophilic wicking material such as a porous plastic having a pore size ranging from approximately 5 microns to approximately 50 microns.

FIG. 1B schematically illustrates the lifetime of tube lines against the vapor pressure of water (P_{H2O}) in prior art tube lines (A), and in the tube lines disclosed herein (B), according to some embodiments. It is understood that as the vapor pressure (humidity) increases the life time of the sampling tube decreases. The normal P_{H2O} typically ranges from about 35 hectoPascal (hPa) to about 70 hPa, however P_{H2O} values as high as 115 hPa can also occur.

As seen in FIG. 1B, the tube disclosed herein, including a groove(s) with uneven side walls, has an extended life time due to the enhanced evaporation achieved through the wall of the tube.

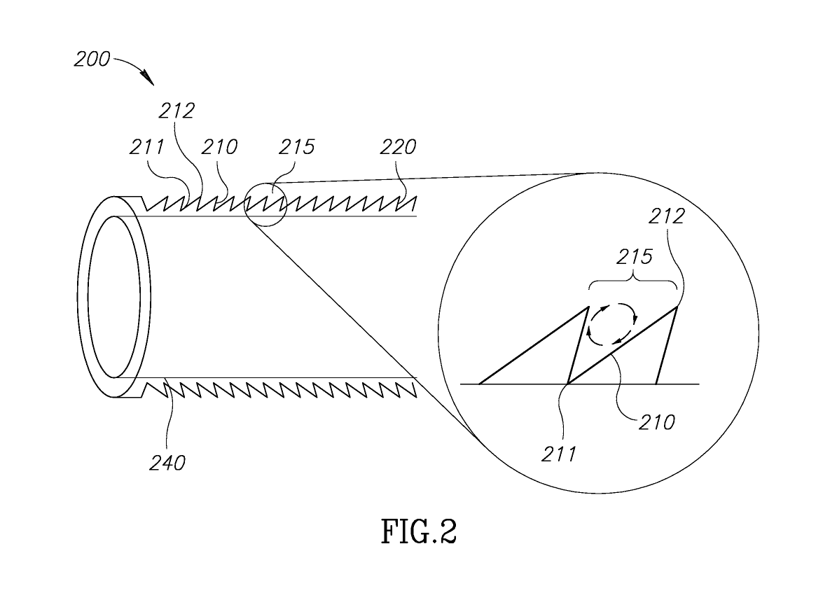

Reference is now made to FIG. 2, which schematically illustrates a tube 200 having an outer wall 220 and an inner wall 240. Groove 210 in outer wall 220 generates a convection driven recirculation zone 215. It is understood by one of ordinary skill in the art, that the temperature of the air is warmest at the bottom 211 of asymmetric grooves 210 (closest to the exhaled breath flowing in tube 200) and coldest at the most distant edge 212 of asymmetric grooves 210. Accordingly, the warmer air in bottom 211 of asymmetric groove 210 is less dense and thus more buoyant than the cooler air on edge 212 of asymmetric grooves 210. In effect, warm air moves upwards while simultaneously being replaced with the cooler air from edge 212. As the warmed air moves away from the exhaled breath flowing in tube 200 it cools down and causes convective movement of the air in effect generating recirculation zone 215 in groove 210.

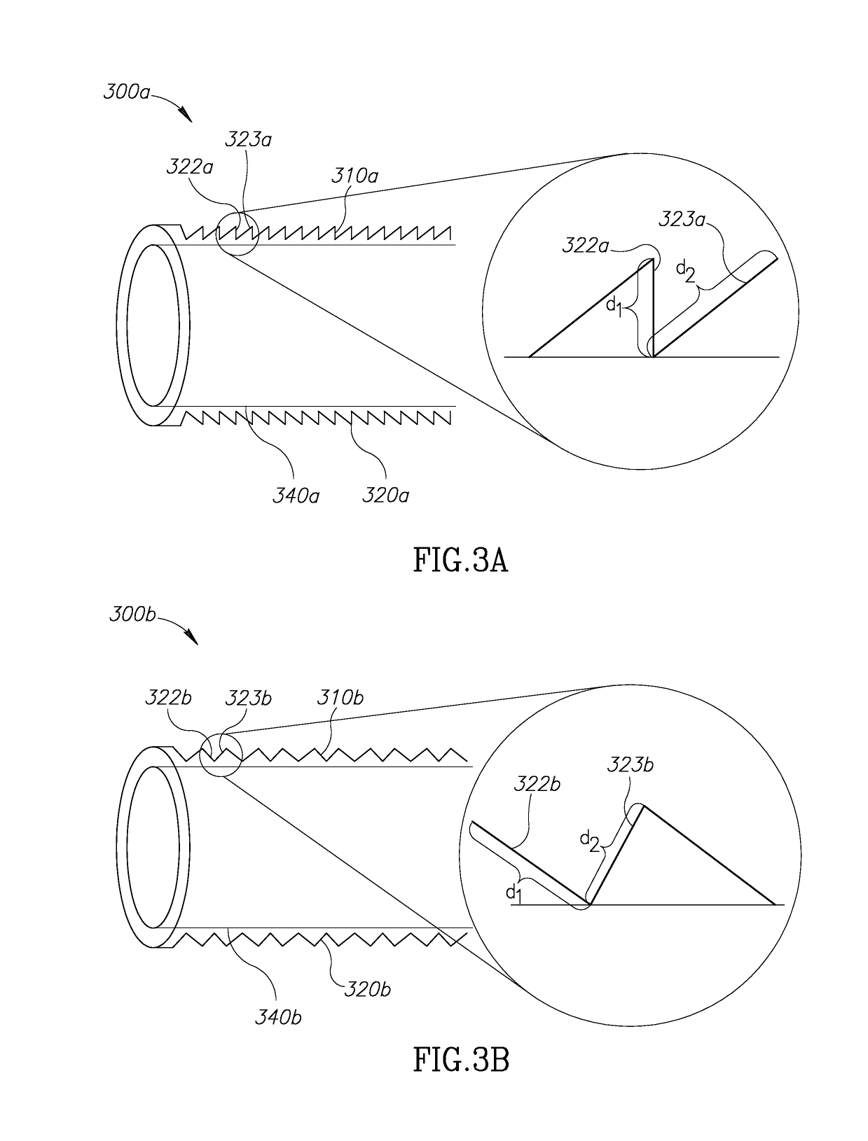

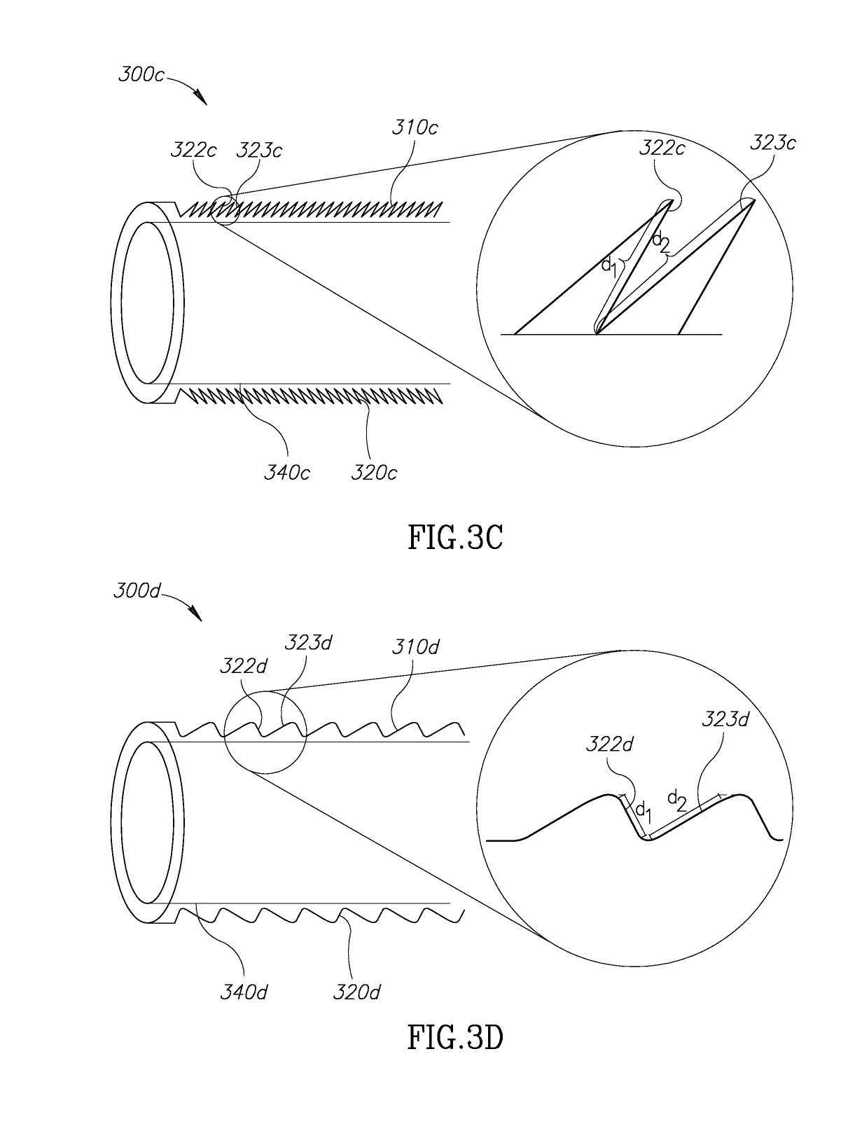

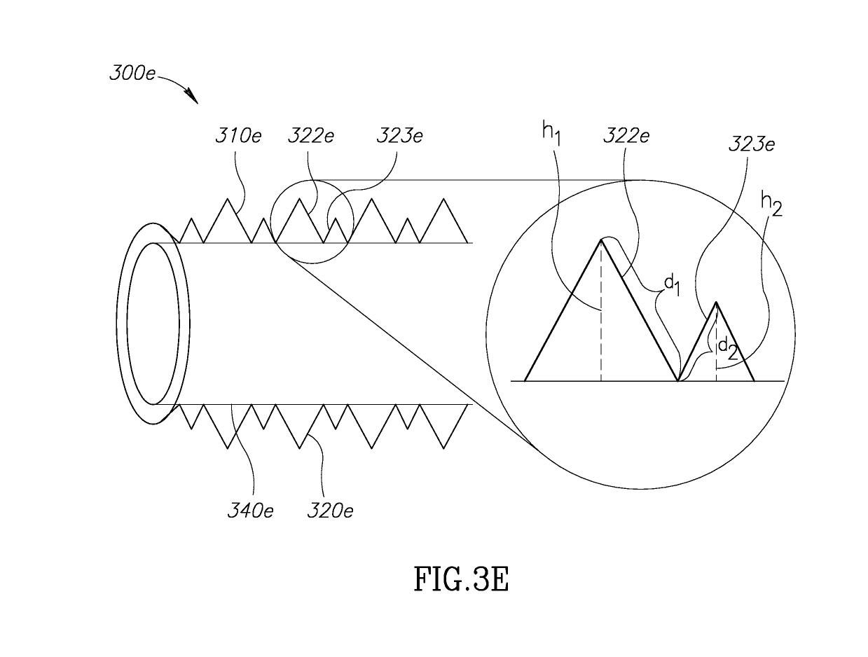

FIG. 3A-E schematically illustrates longitudinal views of parts of breath sampling tubes having grooves with uneven side walls, according to some embodiments. It is understood by one of ordinary skill in the art that the grooves depicted below are representative only and that the number of grooves and/or their distribution along and/or around the tube may vary, for example the grooves may be successive or non-successive, be distributed circumferentially, longitudinally or helically along the entire length of the tube or parts thereof. Such variations in the number and/or distribution of the grooves fall under the scope of this disclosure. It is further clear, that as the number of grooves increases, each groove becomes less distinct and the outer wall of the tubes obtains an overall rough appearing surface. Moreover, according to some embodiments, the grooves having uneven side walls may be replaced by ridges or asymmetric elevations in the outer wall, which likewise serves to produce recirculation zones as essentially described above, and as such fall under the scope of this disclosure.

FIG. 3A schematically illustrates a breath sampling tube 300a having an inner wall 340a and an outer wall 320a. Tube 300a includes groove(s) 310a in outer wall 320a. Groove 310a is formed so as having a first wall 322a, vertical to inner wall 340a and of a length (d1), and a second wall 323a, sloped relative to inner wall 340a and of a length (d2), such that (d1) is shorter than (d2). Sloped walls 322a and 323a may be formed as cuts in outer wall 320a and may thus have a same height.

FIG. 3B schematically illustrates a breath sampling tube 300b having an inner wall 340b and an outer wall 320b. Tube 300b includes groove(s) 310b, in outer wall 320b. Groove 310b is formed so as having a first wall 322b, sloped relative to inner wall 340b, and of a length (d1) and a second wall 323b, sloped in an opposite direction relative to inner wall 340b, and of a length (d2), such that (d1) is longer than (d2). First and second walls 322b and 323b may be formed as cuts in outer wall 320b and may thus have a same height.

FIG. 3C schematically illustrates a breath sampling tube 300c having an inner wall 340c and an outer wall 320c. Tube 300c includes a groove(s) 310c, in outer wall 320c. Groove 310c is formed so as having a first wall 322c, sloped in a same direction relative to inner wall 340c, of a length (d1) and a second wall 323c, sloped relative to inner wall 340c, and of a length (d2), such that (d1) is shorter than (d2). First and second walls 322c and 323c may be formed as cuts in outer wall 320c and may thus have a same height.

FIG. 3D schematically illustrates a breath sampling tube 300d having an inner wall 340d and an outer wall 320d. Tube 300d includes a groove(s) 310d, having a first wall 322d, essentially smoothly sloped relative to inner wall 340d, and a second wall 323d, essentially smoothly sloped relative to inner wall 340d. First and second walls 322d and 323d are formed such that the length (d1) of first wall 322d is shorter than the length (d2) of second wall 323d, as measured from a point closest to inner wall 340d to a point furthest away from inner wall 340d. First and second walls 322d and 323d may be formed as cuts in outer wall 320d and may thus have a same height.

FIG. 3E schematically illustrates a breath sampling tube 300e having an inner wall 340e and an outer wall 320e. Tube 300e includes groove(s) 310e, having a first side wall 322e of a height (h1) and a second side wall 323e of a height (h2); wherein (h1) is higher than (h2). It is understood by one of ordinary skill in the art that, according to alternative embodiments, (h1) may be smaller than (h2). The length (d1) of first wall 322e is here shown as being longer than the length (d2) of second wall 323e. However, it is understood to one of ordinary skill in the art that (d1) and (d2) may also be of a same length (but differently sloped relative to inner wall 340e).

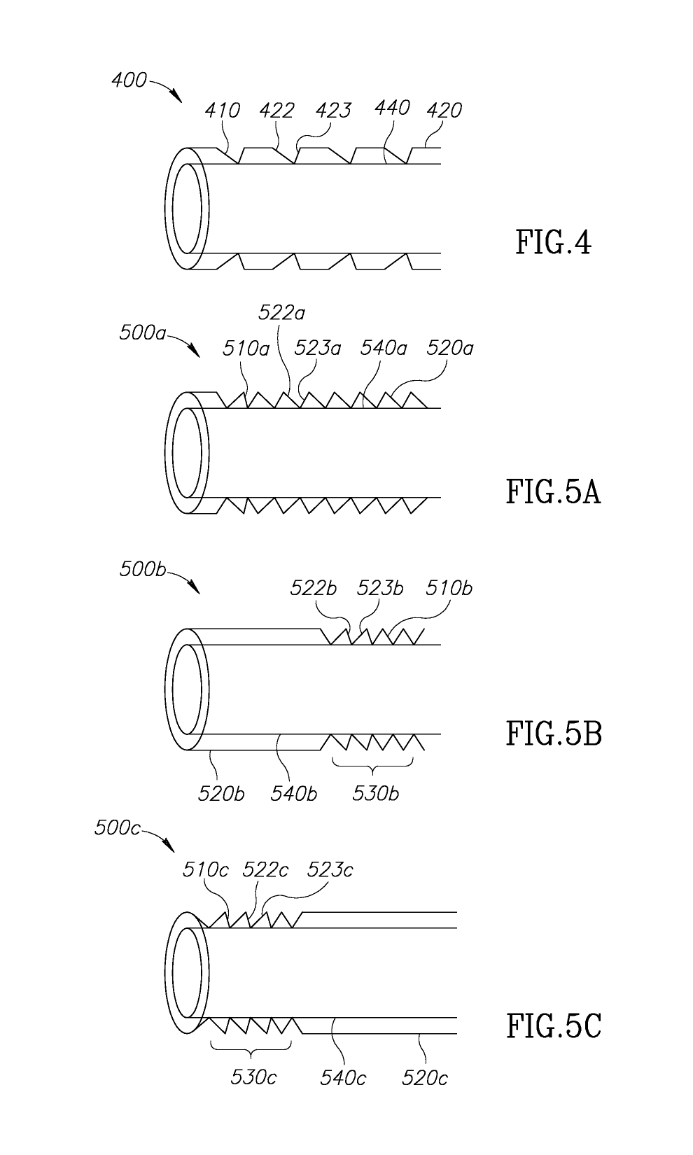

Reference is now made to FIG. 4, which schematically illustrates longitudinal views of parts of breath sampling tubes with grooves having uneven side walls, according to some embodiments. Breath sampling tube 400 has an outer wall 420 and an inner wall 440. Breath sampling tube 400 includes groove 410, with uneven side walls 422 and 423, non-successively distributed in outer wall 420 of tube 400. It is understood that the grooves depicted in FIG. 4 are representative only and that the number of grooves, their distribution and/or configuration along and/or around the sampling tube may vary, for example the grooves may be distributed circumferentially, longitudinally or helically along the entire length of the tube or parts thereof. It is further understood that the grooves in FIG. 4 may be of any configuration, such as, but not limited to the configurations described in FIG. 3A-E above.

Reference is now made to FIG. 5A-E, which schematically illustrates longitudinal views of parts of breath sampling tubes with grooves having uneven side walls, according to some embodiments. It is understood that the grooves depicted in FIG. 5A-E are representative only and that the number of grooves, their distribution and/or configuration along and/or around the sampling tube may vary, for example the grooves may be successive or non-successive, be distributed circumferentially, longitudinally or helically along the entire length of the tube or parts thereof. Such variations in the number, configuration and/or distribution of the grooves fall under the scope of this disclosure. It is further clear, that as the number of grooves increases, each groove becomes less distinct and the outer wall of the tubes obtains an overall rough appearing surface.

FIG. 5A schematically illustrates a breath sampling tube 500a having an outer wall 520a and an inner wall 540a according to some embodiments. Tube 500a includes groove 510a, having uneven side walls 522a and 523a, in outer wall 520a along the entire length of tube 500a.

FIG. 5B schematically illustrates a breath sampling tube 500b having an outer wall 520b and an inner wall 540b according to some embodiments. Tube 500b includes grooves 510b, having uneven side walls 522b and 523b, in an outer wall 520b at a distal end 530b of tube 500b. It is understood that distal end 530b depicted is non-limiting and serves an illustrative purpose only. Distal end 530b may be short, extending a few centimeters, such as for example 2, 3, 4, 5 cm from the end of tube 500b, or longer such as for example 10, 15, 20, 25, 30 cm or more from the end of tube 500b. Each possibility is a separate embodiment.

FIG. 5C schematically illustrates a breath sampling tube 500c having an outer wall 520c and an inner wall 540c according to some embodiments. Tube 500c includes grooves 510c, having uneven side walls 522c and 523c, in an outer wall 520c at a proximal end 530c of tube 500c, according to some embodiments. It is understood that proximal end 530c depicted is non-limiting and serves an illustrative purpose only. Proximal end 530c may be short, extending a few centimeters, such as for example 2, 3, 4, 5 cm from the end of tube 500c, or longer such as for example 10, 15, 20, 25, 30 cm or more from the end. Each possibility is a separate embodiment.

FIG. 5D schematically illustrates a breath sampling tube 500d having an outer wall 520d and an inner wall 540d according to some embodiments. Tube 500d includes grooves 510d, having uneven side walls 522d and 523d, in an outer wall 520d at a central portion 530d thereof, according to some embodiments. It is understood that central portion 530d depicted is non-limiting and serves an illustrative purpose only. Central portion 530d may be short, extending a few centimeters, such as for example 2, 3, 4, 5 cm or longer such as for example 10, 15, 20, 25, 30 cm or more. Each possibility is a separate embodiment.

FIG. 5E schematically illustrates a breath sampling tube 500e having an outer wall 520e and an inner wall 540e according to some embodiments. Tube 500e include sections 530e having a groove(s) 510e with uneven side walls 522e and 523e. It is understood that sections 530e depicted are non-limiting and serve an illustrative purpose only. The length of each section 530e may be short, extending a few centimeters, such as for example 2, 3, 4, 5 cm or longer such as for example 10, 15, 20, 25, 30 cm or more. Each possibility is a separate embodiment. Furthermore, the number of sections 530e may vary from a single section to 2, 3, 4, 5, 10 or more sections. Each possibility is a separate embodiment. According to some embodiments, each tube comprises a plurality of sections.

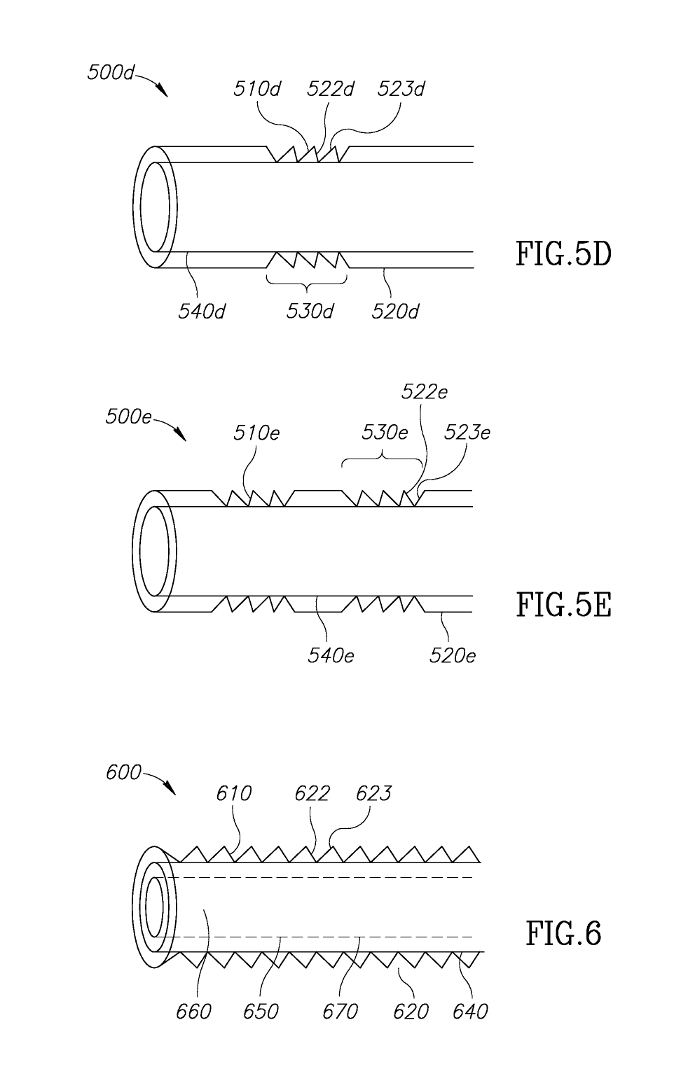

Reference is now made to FIG. 6, which schematically illustrates longitudinal views of parts of breath sampling tubes with grooves having uneven side walls, according to some embodiments. Breath sampling tube 600 has an outer wall 620 and an inner wall 640. Breath sampling tube 600 includes grooves 610, having uneven side walls 622 and 623, in outer wall 620 of tube 600. Grooves 610 may be of any configuration, such as, but not limited to the configurations described in FIG. 3A-E above. Breath sampling tube 600 further includes an inner conduit 650 configured to permit gas flow along a central portion 660 of conduit 650 and to store liquids along a surface 670 of conduit 650. Optionally, surface 670 of inner conduit 650 include a hydrophilic material. The liquids flowing in inner conduit 650 are then repelled through surface 670, away from central portion 660 of inner conduit 650. Grooves 610, with uneven side walls 622 and 623, then facilitate enhanced evaporation of the repelled liquids, leaving central portion 660 of inner conduit 650 free for the passage of the exhaled breath sample. Grooves 610 are here illustrated to extend along the entire length of tube 600, however the distribution of grooves 610 may be according to any of the embodiments described above as well as other suitable distributions.

It is understood that the grooves having uneven side walls, illustrated in FIG. 6, may be of any configuration and distribution and that the number of grooves may vary, for example the grooves may be successive or non-successive, be distributed circumferentially, longitudinally or helically along the entire length of the tube or parts thereof. Such variations in the number, configuration and/or distribution of the grooves fall under the scope of this disclosure. It is further clear, that as the number of grooves increases, each groove becomes less distinct and the outer wall of the tubes obtains an overall rough appearing surface.

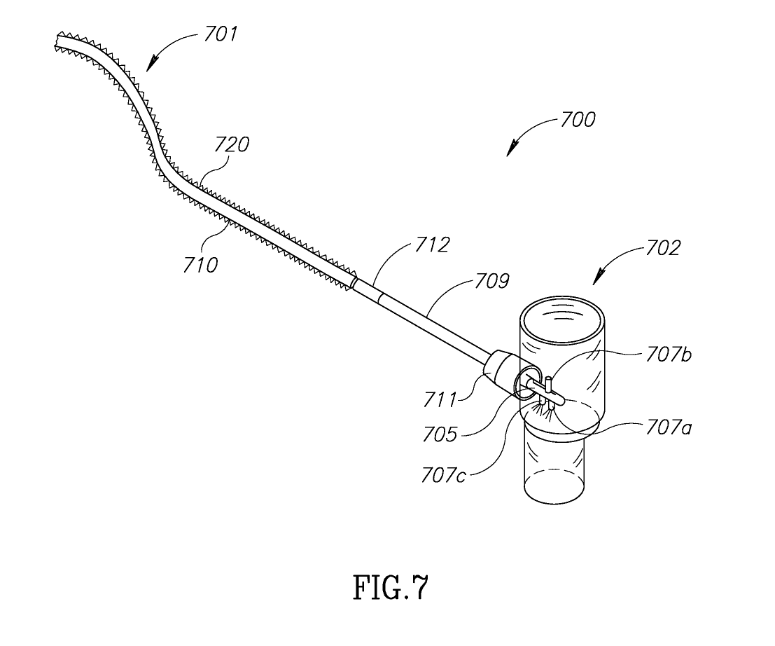

Reference is now made to FIG. 7 schematically illustrates a breath sampling system 700 including a breath sampling tube 701 including groove 710, in an outer wall 720 thereof; and a connector 711, according to some embodiments. Breath sampling tube 701 may essentially correspond to any of the tubes described herein and grooves 710 may be of any configuration, such as, but not limited to the configurations described in FIG. 3A-E above. Breath sampling system 700 is configured to enhance the evaporation rate of liquids thereby extending the life time of the tube as well as preventing liquids from reaching sensitive analyzing equipment, such as for example a capnograph (not shown). Exhaled breath sample collection is done through an airway adapter, such as airway adapter 702, which may be essentially a tube with connector fittings at each end which may be adapted to a patient airway tubing. Airway adapter 702 may comprise at least one sampling port, such as sampling port 705. Sampling port 705 may have at least one sampling inlet, such as sampling inlets 707a-c through which the exhaled and inhaled breath sample is collected and passed into breath sampling system 700. The exhaled breath sample collected in airway adapter 702 may be passed through sampling port 705 into breath sampling tube 701. Groove(s) 710 in outer wall 720 of breath sampling tube 701 are configured to enhance the evaporation rate of liquids from breath sampling tube 701, without interfering with the waveform of the sample. Optionally, breath sampling tube 701 may also include an inner conduit (not shown), as essentially described above. According to some embodiments, breath sampling tube 701 may be connected directly to airway adapter 702 via a connector such as connector 711 (option not shown), such that the exhaled breath sample enters breath sampling tube 701 directly from breath sampling port 705 and further on to the gas analyzer (not shown). Alternatively, breath sampling system 700 may also include a moisture reduction system (MRS) 709, configured to reduce liquids from entering breath sampling tube 701. MRS 709 may at one end thereof be connected to airway adapter 702 via connector 711 and at the other end thereof to breath sampling tube 701 via connector 712. It is understood by one of ordinary skill in the art that MRS 709 may be a specially designed tube, which may be of variable length and diameter, which may include any drying mechanism and/or material, essentially impermeable to gas, that is capable of reducing moisture level, for example Nafion®, and/or filters such as micro-porous filters or molecular sieves (material containing tiny pores that may be used to absorb moisture.

Referring to FIG. 7, the following is a description of the operation of breath sampling system 700 according to some embodiments. A patient is connected to a breathing apparatus or to some other ventilation means through a breathing tube or patient airway tube to which is adapted an airway adapter, such as airway adapter 702. Samples of exhaled breath from the patient, which may include liquid secretions such as blood, mucus, water, medications, and the like, are sucked into sampling inlets, such as sampling inlets 707a-c and into sampling port 705, typically by means of negative pressure supplied by a pumping element (not shown) which may be connected to breath sampling tube 701. The breath sample (including the liquid secretions) may optionally pass from sampling port 705 into MRS 709 where moisture is extracted from the exhaled breath samples. MRS 709 is placed as close as possible to airway adapter 702 so as to immediately try to counteract the effects of the liquids in the exhaled breath samples which may contribute to clogging in breath sampling tube 701. Although MRS 709 is able to extract a good portion of the moisture and liquids, significant amounts may remain in the exhaled breath samples which may hamper the accurate monitoring and analysis of the samples by the measurement sensor in addition to possibly blocking the path of the flow of the samples in breath sampling system 700. However, groove(s) 710 in outer wall 720 of breath sampling tube 701 enhance the evaporation of the liquids from the surface of breath sampling tube 701 and thereby prevent liquid accumulation in the tube.

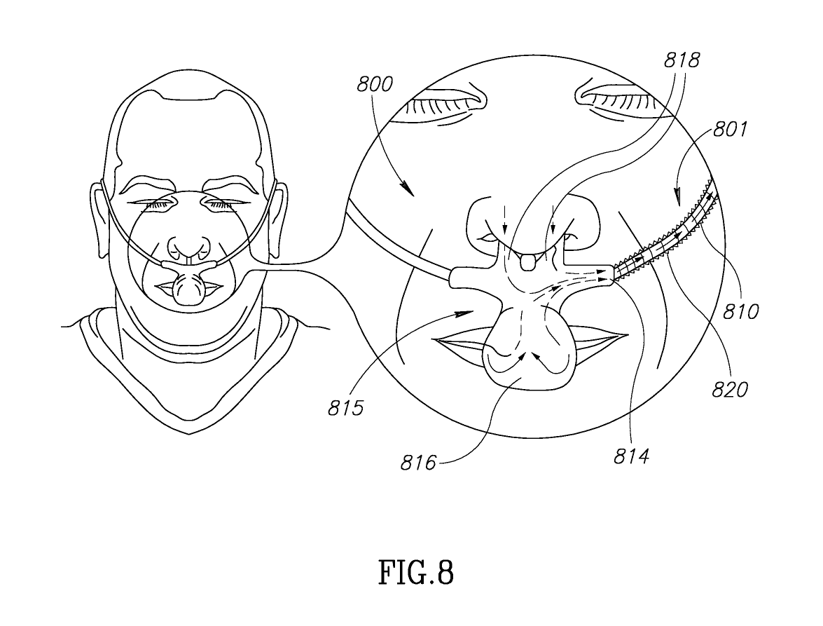

Reference is now made to FIG. 8 which schematically illustrates a breath sampling system 800 including a breath sampling tube 801 including a groove(s) 810 in an outer wall 820 thereof; and an oral nasal cannula, such as oral/nasal cannula 815 according to some embodiments. Breath sampling tube 801 may essentially correspond to any of the tubes described herein; and grooves 810 may be of any configuration, such as, but not limited to the configurations described in FIG. 3A-E above. Breath sampling system 800 is configured to enhance the evaporation rate of liquids thereby extending the life time of the tube as well as preventing liquids from reaching sensitive analyzing equipment, such as for example a capnograph (not shown). Breath exhaled through the subject's nose is directed through nasal prongs 818 toward an exhaled breath collection bore 814. In a similar manner, breath exhaled through the subject's mouth is collected in oral scoop element 816, and is directed to exhaled breath collection bore 814. The exhaled breath collected in exhaled breath collection bore 814 flows into breath sampling tube 801, typically by means of negative pressure supplied by a pumping element (not shown), which may be connected to breath sampling tube 801. Groove(s) 810 of breath sampling tube 801 are configured to enhance the evaporation rate of liquids from breath sampling tube 801, without interfering with the waveform of the sample.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A breath sampling tube comprising an outer wall and an inner wall, wherein said outer wall comprises at least one groove, said groove having uneven side walls arranged to induce a convection driven circulation zone in said at least one groove; wherein at least part of said tube is formed of a material configured to evaporate liquid.

2. The tube according to claim 1, wherein said uneven side walls comprise a first side wall and a second side wall, wherein a length (dl) of said first side wall is different from a length (d2) of said second side wall.

3. The tube according to claim 1, wherein (d1)>(d2).

4. The tube according to claim 1, wherein (d1)<(d2).

5. The tube according to claim 1, wherein said at least one groove is formed circumferentially around said tube.

6. The tube according to claim 1, wherein said outer wall comprises a plurality of grooves having uneven side walls.

7. The tube according to claim 1, wherein said at least one groove generates a rough surface in said outer wall.

8. The tube according to claim 1, wherein said at least one groove enhance airflow instability along said outer wall of said tube.

9. The tube according to claim 1, wherein said at least one groove enhances the evaporation of liquids from said tube, thereby extending the life time of said tube.

10. The tube according to claim 1, wherein said material configured to evaporate liquids is a hydrophilic material.

11. The tube according to claim 1, wherein said outer wall comprises a hydrophilic material.

12. The tube according to claim 1, wherein said inner wall comprises a hydrophilic material.

13. The tube according to claim 1, further comprising an inner conduit.

14. The tube of claim 13, wherein said inner conduit is configured to permit gas flow along a central portion of said conduit and to store liquids along a surface of said conduit.

15. The tube of claim 13, wherein the surface of said inner conduit comprises a hydrophilic material.

16. A breath sampling system comprising:
a breath sampling tube comprising an outer wall and an inner wall wherein at least part of the outer wall comprises at least one groove, said groove having uneven side walls arranged to induce a convection driven circulation zone in said at least one groove; wherein at least part of said tube is formed of a material configured to evaporate liquids; and
at least one connector.

17. A method comprising forming a breath sampling tube comprising an outer wall and an inner wall, wherein at least a part of the outer wall comprises at least one groove, the groove having uneven side walls arranged to induce a convection driven circulation zone in the at least one groove.

18. The method of claim 17, wherein at least part of the tube is formed of a material configured to evaporate water.

19. The method of claim 18, wherein the uneven side walls comprise a first side wall and a second side wall, wherein a length (d1) of the first side wall is different from a length (d2) of the second side wall.
